(19) United States
(12) Plant Patent Application Publication
(10) Pub. No.: US 2025/0338782 P1
(43) Pub. Date: Oct. 30, 2025

(54) Raspberry Plant Named ‘PS-12.082-54’

(50) ;

(71) Applicant: Plant Sciences, Inc., Watsonville, CA (US)

(72) Inventor: Scott W. Adams, Carmel Valley, CA (US)

(73) Assignee: Plant Sciences, Inc., Watsonville, CA (US)

(21) Appl. No.: 19/175,880

(22) Filed: Apr. 10, 2025

Related U.S. Application Data
(60) Provisional application No. 63/638,749, filed on Apr. 25, 2024.

(51) Int. Cl. A01H 6/74; A01H 5/08

(52) U.S. Cl. CPC A01H 6/7499; A01H 5/08
USPC PLT/204

(57) ABSTRACT

This invention relates to a new and distinct everbearing variety of raspberry plant named ‘PS-12.082-54’. The new variety is primarily adapted to the growing conditions of the central coast of California and is characterized by the following: mid-season primocane production with medium sized fruit of strong yellow orange coloration. Fruit is of consistent conic shape, releases easily from receptacle, is very glossy and possesses good flavor. Foliage is flat, medium green; possessing weak rugosity, weak gloss and mostly 3 foliates. Primocanes have an absent or very weak waxy coat, medium thorn density of small green prickles and are absent of anthocyanin coloration.

Latin name of the genus and species of the plant claimed: Rubus idaeus.

Variety denomination: ‘PS-12.082-54’.

### CROSS-REFERENCE TO RELATED APPLICATION

This application claims priority from U.S. Provisional Patent Application No. 63/638,749, filed Apr. 25, 2024. The contents of this application are incorporated herein by reference in their entirety.

### BACKGROUND OF THE INVENTION

The present invention relates to a new and distinct fall-bearing raspberry variety designated as ‘PS-12.082-54’. This new variety is a result of a controlled cross made in 2012 in Watsonville, California between raspberry variety ‘BOUNTIFUL’ (patented, U.S. Plant Pat. No. 27,582) as the female parent and raspberry variety ‘PARAGON’ (patented, U.S. Plant Pat. No. 27,583) as the male parent in an ongoing breeding program. The variety is botanically known as Rubus idaeus and was tested as ‘RR 12.082-54’ and ‘PS-8254’.

The seedling resulting from the aforementioned cross was asexually propagated by dormant canes in Santa Cruz County, California and was subsequently selected by the inventor from a controlled breeding plot in Watsonville, California in 2014. After its selection, the new variety was further asexually propagated by dormant canes, roots and non-dormant root shoot cuttings in Watsonville (Santa Cruz County), Manteca (San Joaquin County), and Macdoel (Siskiyou County), California. The new variety was then extensively tested over the next several years in fruiting fields in Watsonville, California. This propagation has demonstrated that the combination of traits disclosed herein as characterizing the new variety are fixed and remain true to type through successive generations of asexual reproduction.

BRIEF SUMMARY OF THE INVENTION

‘PS-12.082-54’ is primarily adapted to the climate and growing conditions of the central coast of California. This region provides the necessary year-round temperatures required for it to produce and maintain a strong vigorous plant and to remain in fruit production from July through December on primocanes and in the ensuing year from May through July on the floricanes. Damage to fruit and young leaves has been observed in summer temperatures at or above 95° F. (35° C.). No winter damage has been observed on fruiting plants in off-cycle (winter) production regions of USDA climate zones 9b and above. No winter damage has been observed on dormant plants in on-cycle (summer) production regions of USDA climate zones 7b-9b. In climate zones below this, winter hardiness is unknown for ‘PS-12.082-54’. ‘PS-12.082-54’ is not drought tolerant and requires frequent irrigation to maintain proper plant health. The following traits have been repeatedly observed and are determined to be unique characteristics of ‘PS-12.082-54’, which in combination distinguish this raspberry plant as a new and distinct variety:
1. Strong yellow green prickles
2. High fruit yield
3. Strong orange yellow fruit
4. Absent to very weak primocane glaucosity
5. Good flavor

The raspberry variety that is believed to be most closely related to the new raspberry variety ‘PS-12.082-54’ is the raspberry variety ‘MAJESTIC’ (patented, U.S. Plant Pat. No. 31,716). In comparison to the similar raspberry variety ‘MAJESTIC’, ‘PS-12.082-54’ differs by the following combination of characteristics described in Table 1:

### Comparison Between ‘PS-12.082-54’ and ‘Majestic’

TABLE 1

| | | ‘MAJESTIC’ |
|---|---|---|
| Characteristic | ‘PS-12.082-54’ | (US PP31,716) |
| Prickle coloration (tip) | RHS N144D | RHS N186B |
| | Strong yellow green | Dark greyish purple |
| Leaf shape (cross section) | Flat | Slightly convex |
| Rugosity | Weak | Medium |
| Color mature fruit | RHS 17A | RHS 43A |
| | Strong orange yellow | Vivid reddish orange |
| Relative size of drupes | Large | Medium |
| Predominant shape of fruit | Conical | Conical |
| Peduncle anthocyanin | Absent | Absent to very weak |
| intensity | | |
| Relative number | 19.6 | 22.1 |
| of pedicel prickles | Medium | Many |

‘PS-12.082-54’ differs from its parents, ‘BOUNTIFUL’ and ‘PARAGON’ by the following combination of characteristics described in Table 2:

### Comparison Between ‘PS-12.082-54’, ‘Bountiful’, and ‘Paragon’

TABLE 2

| | ‘PS- | ‘BOUNTIFUL’ | ‘PARAGON’ |
|---|---|---|---|
| Characteristic | 12.082-54’ | (US PP27,582) | (US PP27,583) |
| Productivity | High | High | Medium |
| Crates/acre (18-mo.) | 10,892 | 10,476 | 7,222 |
| Color mature fruit | RHS 17A | RHS 34A | RHS 34A |
| | Strong yellow | Vivid reddish | Vivid reddish |
| | orange | orange | orange |
| Glossiness (fruit) | Very high | Medium | High |
| Firmness (fruit) | High | High | Very high |
| Adherence of receptacle | Weak | Very weak | Medium |
| Primocane time | Medium | Late | Early |
| of fruiting | | | |
| Relative size of drupelet | Large | Medium | Small |
| Primocane fruit | 4.1 | 4.6 | 4.2 |
| weight (g) | | | |
| Flavor | Good | Good | Excellent |
| Soluble solids (% brix) | 10.2 | 9.8 | 11.6 |

For identification, a series of molecular markers have been determined for this new variety.

### BRIEF DESCRIPTIONS OF THE DRAWINGS

The accompanying color photographs illustrate the overall appearance of typical specimens of the new raspberry variety ‘PS-12.082-54’ at various stages of development as true as reasonably possible with color reproductions of this type. Color in the photographs may differ slightly from the color value cited in the detailed botanical description which accurately describes the color of ‘PS-12.082-54’. The depicted plant and plant parts of the new raspberry variety ‘PS-12.082-54’ were taken in Watsonville, California and are approximately 2 to 16 months old:

FIG. 1 shows typical primocane fruit shape, color and gloss characteristics of ‘PS-12.082-54’ taken in the month of August 2024;

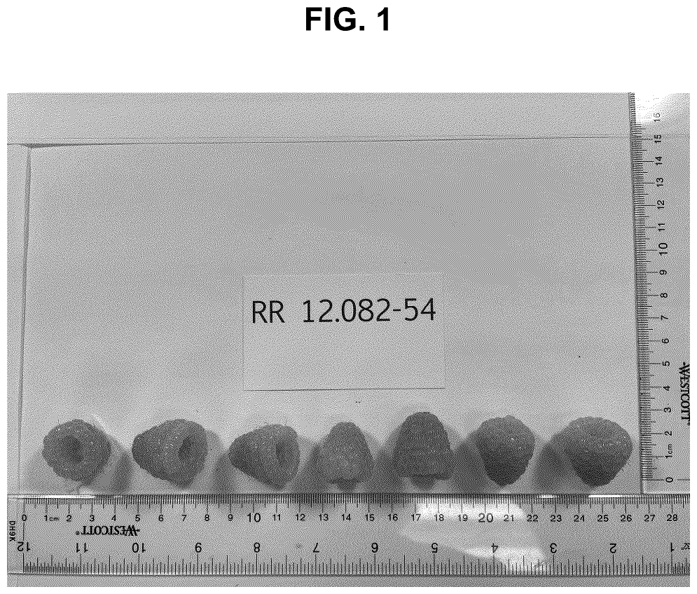

FIG. 2 shows typical shape, gloss, coloration, foliate and rugosity characteristics of of ‘PS-12.082-54’ taken in the month of August 2024;

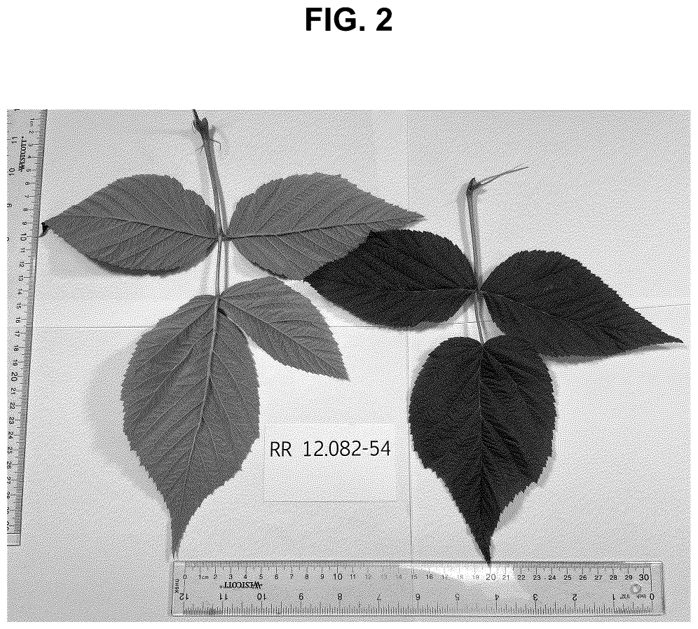

FIG. 3 shows typical young shoot characteristics of ‘PS-12.082-54’ taken in the month of March 2025;

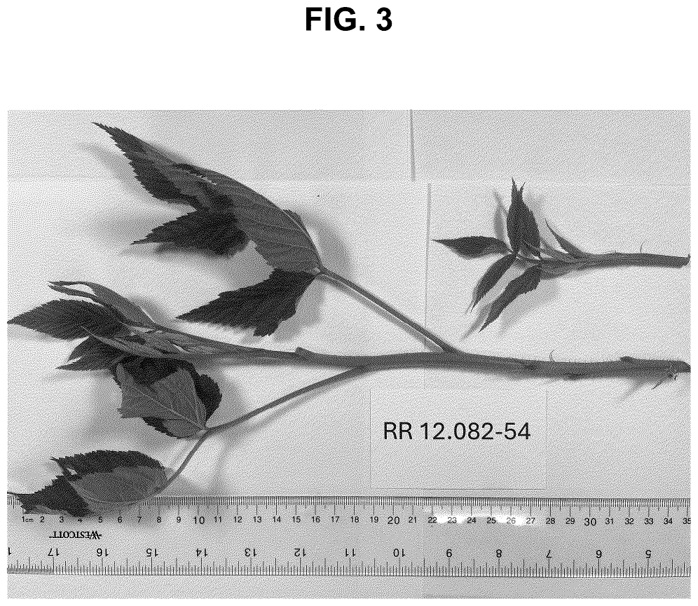

FIG. 4 shows typical floricane characteristics of ‘PS-12.082-54’ taken in the month of March 2025.

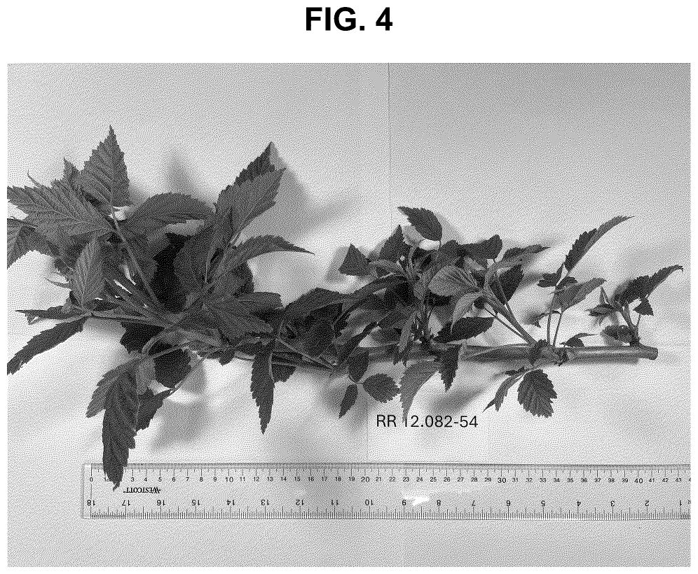

FIG. 5 shows typical floricane characteristics of ‘PS-12.082-54’ taken in the month of May 2022.

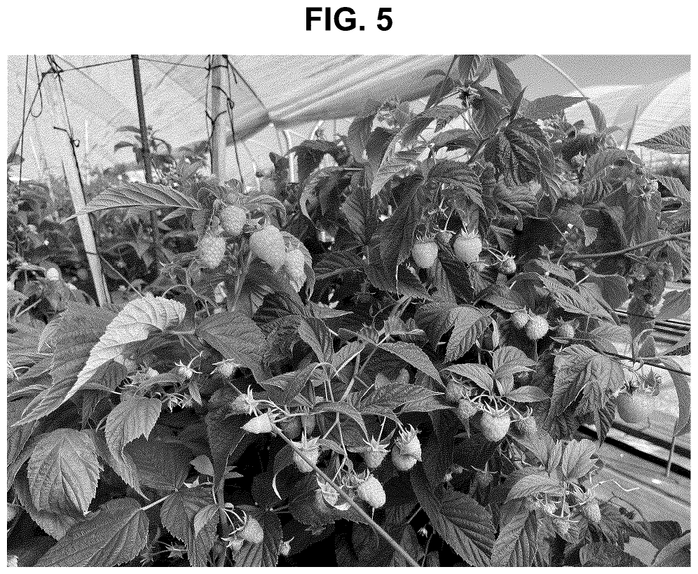

### DETAILED BOTANICAL DESCRIPTION

‘PS-12.082-54’ has not been observed under all possible environmental conditions. The characteristics of the new variety may vary in detail, depending upon variations in environmental factors, including weather (temperature, humidity and light intensity), day length, soil type, and location.

The aforementioned photographs, together with the following description of the new raspberry variety ‘PS-12.082-54’, unless otherwise noted, are based upon observations taken during the 2023-2024 growing season in Watsonville, California. Primocane measurements and ratings were taken from plants of ‘PS-12.082-54’ dug from a nursery located in Siskiyou County, California during the middle of October 2022 and planted approximately 3 to 4 weeks later in Watsonville, California. The approximate age of the observed primocane plants is 7 to 8 months. Floricane measurements and ratings were taken from the same planting of ‘PS-12.082-54’ at an approximate age of 16 to 18 months. Yield observations and fruit quality characteristics are averaged from 3 years of data collected from the 2021 through 2024 production seasons. Flower measurements and characteristics are from secondary flowers unless otherwise noted. Fruit characteristics and measurements are from secondary fruit unless otherwise noted. Foliage characteristics and measurements are from 3-foliate foliage unless otherwise noted.

Color terminology where noted follows The R.H.S Colour Chart Sixth Edition, Royal Horticultural Society, London, United Kingdom (2015).

The following Tables 3-7 describe fruit, plant, foliage, flower and pest/disease characteristics of the new raspberry ‘PS-12.082-54’ in comparison to the similar raspberry variety ‘MAJESTIC’ (patented, U.S. Plant Pat. No. 31,716).

### Comparison Between ‘PS-12.082-54’ and ‘Majestic’

TABLE 3
FRUIT CHARACTERISTICS

| | | ‘MAJESTIC’ |
|---|---|---|
| Characteristic | ‘PS-12.082-54’ | (US PP31,716) |
| Color mature fruit | RHS 17A | RHS 43A |
| | Strong orange yellow | Vivid reddish orange |
| Color achenes | RHS 159A | RHS 159A |
| | Light yellowish pink | Light yellowish pink |
| Fruit length (mm) | 26.22 | 25.49 |
| Fruit width (mm) | 22.74 | 22.73 |
| Length/width ratio | 1.15 | 1.12 |
| | Longer than broad | Longer than broad |
| Seed weight (mg) | 1.55 | 1.68 |
| Drupelets per berry | 107 | 131 |
| Weight of single | 0.066 | 0.044 |
| drupe (g/drupe) | | |
| Drupe length (mm) | 4.44 | 7.15 |
| Drupe diameter (mm) | 4.87 | 4.19 |
| Relative size of drupes | Large | Medium |
| Fruit size | Medium | Medium |
| Fruit per node | 12 | 11 |
| Predominant shape | Conical | Conical |
| Receptacle length (mm) | 24.04 | 23.55 |
| Receptacle diameter (mm) | 10.22 | 10.41 |
| Receptacle color | RHS 155B | RHS 155B |
| | Pale yellow | Yellowish white |
| Evenness of color (fruit) | Even | Even |
| Glossiness | Very high | High |
| Adherence of receptacle | Weak | Weak |
| Firmness of flesh | Medium | High |
| Firmness of skin | High | High |
| Soluble solids (% brix) | 10.2 | 10.8 |
| Flavor | Good | Very good |
| Fruit use (intended market) | Fresh market | Fresh market |

TABLE 4
PLANT CHARACTERISTICS

| | | ‘MAJESTIC’ |
|---|---|---|
| Characteristic | ‘PS-12.082-54’ | (US PP31,716) |
| General: | | |
| Habit | Upright | Upright |
| Plant height (m) | 1.4 | 1.8 |
| Plant width (cm) | 31 | 35 |
| Time to initiate roots (days) | 10-12 | 10-12 |
| Canes per hill | 10.5 | 10.4 |
| Productivity | High | High |
| Crates/acre (18-mo.) | 10,892 | 10,490 |
| Self-fruitfulness | Yes | Yes |
| Type of bearing | Everbearing | Everbearing |
| Primocane: | | |
| Color (true) | RHS N144C | RHS 145A |
| | Strong yellow green | Strong yellow green |
| Length (cm) | 144.2 | 178.1 |
| Basal diameter (mm) | 20.77 | 13.62 |
| Diameter central ⅓ (mm) | 10.98 | 11.38 |
| Lateral length | 30.5 | 28.5 |
| at central ⅓ (cm) | | |
| No. fruiting laterals per cane | 15.7 | 15.3 |
| Total nodes per cane | 36 | 35 |
| Internode length | 44.08 | 39.01 |
| at central ⅓ (mm) | | |
| Anthocyanin coloration | n/a | RHS 59B |
| | | Deep purplish red |
| Anthocyanin intensity | Absent | Absent to very weak |
| Pubescence | Absent | Absent |
| Vegetative bud length (mm) | 6.25 | 10.34 |
| Vegetative bud | 4.88 | 5.46 |
| diameter (mm) | | |
| Vegetative bud shape | Widely ovate | Ovate |
| Vegetative bud color | RHS 166A | RHS 187B |
| | Greyish brown | Dark red |
| Strength of waxy | Absent to very weak | Absent to very weak |
| coat (glaucosity) | | |
| Time of flowering | Medium | Medium |
| Time of fruiting | Medium | Medium |
| Length of fruiting season | Long | Long |
| % of total yield | 69% | 44% |
| Flowering period | Late June to | Late June to |
| | Late November | Late November |
| Harvest period | Late July to | Late July to |
| | Late December | Late December |
| Primocane fruit weight (g) | 4.1 | 4.2 |
| Primocane yield (g/plant) | 3,655 | 2,571 |
| Young Shoots: | | |
| Number (per meter) | 20-25 | 15-20 |
| | Medium | Medium |
| Anthocyanin presence | Absent | Present |
| Anthocyanin coloration | n/a | RHS 179A |
| | | Moderate red |
| Anthocyanin intensity | Absent | Medium |
| Prickles: | | |
| Prickle presence | Present | Present |
| Prickle coloration (tip) | RHS N144D | RHS N186B |
| | Strong yellow green | Dark greyish purple |
| Prickle coloration (base) | RHS N144C | RHS N186C |
| | Strong yellow green | Dark greyish red |
| Prickle length at | 0.52 | 0.70 |
| central ⅓ (mm) | | |
| Prickle base at | 0.41 | 1.23 |
| central ⅓ (mm) | | |
| Prickle density | 5.12 | 5.35 |
| per cm at central ⅓ | Medium | Medium |
| Prickle texture | Rigid | Rigid |
| Attitude of the tip | Horizontal | Horizontal |
| Floricane: | | |
| Color (true) | RHS N167A | RHS 165B |
| | Brownish orange | Brownish orange |
| Length (cm) | 107.4 | 130.8 |
| Width at central ⅓ (mm) | 9.75 | 10.88 |
| Total nodes per cane | 16 | 20 |
| Internode length | 44.77 | 40.70 |
| at central ⅓ (mm) | | |
| Fruiting lateral attitude | Erect | Erect |
| Time bud burst | Medium | Medium |
| Time of flowering | Medium | Medium |
| Time of fruiting | Medium | Medium |
| Length of fruiting season | Medium to long | Medium to long |
| % of total yield | 31% | 56% |
| Flowering period | Late April to | Late April to |
| | late June | late June |
| Harvest period | Late May to | Late May to |
| | late July | late July |
| Floricane fruit weight (g) | 4.0 | 4.0 |
| Floricane yield (g/plant) | 1,479 | 3,273 |

TABLE 5
FOLIAGE CHARACTERISTICS

| | | ‘MAJESTIC’ |
|---|---|---|
| | ‘PS-12.082-54’ | (US PP31,716) |
| Characteristic | (3 Foliate) | (3 Foliate) |
| General: | | |
| Color of upper surface | RHS NN137B | RHS 137A |
| | Greyish olive green | Moderate olive green |
| Color of lower surface | RHS 190B | RHS 192A |
| | Pale green | Pale yellow green |
| Venation | Pinnate | Pinnate |
| Shape in cross section | Flat | Slightly convex |
| Arrangement | Compound | Compound |
| Relief between | Weak | Medium |
| veins (rugosity) | | |
| Glossiness | Weak | Weak |
| Number of leaflets/leaf | Mostly 3 | Equal 3-5 |
| Total leaf length (cm) | 23.4 | 21.2 |
| Total leaf width (cm) | 22.8 | 22.1 |
| Terminal Leaflet: | | |
| Length (mm) | 143.9 | 116.2 |
| Width (mm) | 87.4 | 79.1 |
| Length/width ratio | 1.6 | 1.5 |
| | Longer than broad | Longer than broad |
| Size | Medium | Medium |
| Shape | Cordate | Cordate |
| Shape of base | Rounded | Cordate |
| Shape of tip | Acuminate | Acuminate |
| Margins | Biserrate | Biserrate |
| Lateral Leaflet: | | |
| Length (mm) | 113.8 | 104.9 |
| Width (mm) | 56.8 | 77.2 |
| Length/width ratio | 2.0 | 1.3 |
| | Much longer | Longer than broad |
| | than broad | |
| Rachis length (mm) | 36.4 | 43.1 |
| Rachis diameter (mm) | 1.54 | 1.90 |
| Rachis anthocyanin | n/a | RHS 184A |
| coloration | | |
| of upper surface | | Greyish red |
| Rachis anthocyanin intensity | Absent | Weak |
| Orientation | Opposite | Opposite |
| Arrangement | Compound | Compound |
| Shape | Ovate | Ovate |
| Overlapping | Free | Overlapping |
| Shape of the base | Oblique | Rounded |
| Shape of the tip | Acuminate | Acuminate |
| Margins | Biserrate | Biserrate |
| Petiole: | | |
| Length (mm) | 73.68 | 61.10 |
| Width (mm) | 2.81 | 4.68 |
| Prickle presence | Yes | Yes |
| Prickle orientation | Erect | Erect |
| Anthocyanin coloration | n/a | RHS 180B |
| of upper surface | | Moderate red |
| Anthocyanin intensity | Absent | Absent to very weak |
| of upper surface | | |
| Stipule length (mm) | 8.34 | 10.70 |
| Stipule width (mm) | 0.62 | 0.89 |
| Stipule orientation | Erect | Erect |

TABLE 6
FLOWER CHARACTERISTICS

| | | ‘MAJESTIC’ |
|---|---|---|
| Characteristic | ‘PS-12.082-54’ | (US PP31,716) |
| Petal color (upper surface) | RHS NN155A | RHS 155C |
| | Yellowish white | Greenish white |
| Petal color (lower surface) | RHS NN155A | RHS 155C |
| | Yellowish white | Greenish white |
| Flower diameter (mm) | 22.24 | 21.22 |
| Petal Length (mm) | 7.71 | 7.46 |
| Petal width (mm) | 3.15 | 3.10 |
| Petal length/width ratio | 2.44 | 2.40 |
| | Much longer | Much longer |
| | than broad | than broad |
| Petal shape in cross section | Flat | Flat |
| Petal shape (overall) | Narrowly spatulate | Narrowly spatulate |
| Petal shape (apex) | Rounded | Rounded |
| Petal shape (base) | Attenuate | Attenuate |
| Petal margin | Entire | Entire |
| Petal texture | Glabrous | Glabrous |
| No. petals/flower | 5.2 | 5.2 |
| No. sepals/flower | 5.2 | 5.4 |
| Sepal length (mm) | 9.26 | 9.70 |
| Sepal width at base (mm) | 5.13 | 5.20 |
| Sepal shape | Widely deltate | Widely deltate |
| Sepal shape (apex) | Acuminate | Acuminate |
| Sepal coloration | RHS 144A | RHS 144A |
| (upper surface) | Strong yellow green | Strong yellow green |
| Sepal coloration | RHS 144B | RHS 144B |
| (lower surface) | Strong yellow green | Strong yellow green |
| Sepal margin | Entire | Entire |
| Sepal texture | Slightly pubescent | Slightly pubescent |
| No. stamen/flower | 82 | 95 |
| Filament length (mm) | 5.08 | 5.19 |
| Filament color | RHS 155C | RHS 155C |
| | Greenish white | Greenish white |
| Pollen quantity | Medium | High |
| Pollen color | RHS 196D | RHS 196D |
| | Yellowish white | Yellowish white |
| Anther length (mm) | 1.20 | 1.12 |
| Anther diameter (mm) | 0.48 | 0.51 |
| Anther coloration | RHS 155C | RHS 155C |
| (pre-dehiscence) | Greenish white | Greenish white |
| Anther coloration | RHS 165B | RHS 165B |
| (post-dehiscence) | Brownish orange | Brownish orange |
| Stigma shape | Lobed | Lobed |
| Stigma length (mm) | 0.11 | 0.10 |
| Stigma diameter (mm) | 0.40 | 0.41 |
| Stigma coloration | RHS 145D | RHS 145D |
| | Light yellow green | Light yellow green |
| Style length (mm) | 3.09 | 3.10 |
| Style diameter (mm) | 0.40 | 0.40 |
| Style coloration | RHS 145D | RHS 145D |
| | Light yellow green | Light yellow green |
| Ovule length (mm) | 1.11 | 1.39 |
| Ovule diameter (mm) | 1.01 | 1.42 |
| Ovule coloration | RHS 145B | RHS 145B |
| | Light yellow green | Light yellow green |
| Pedicle length (mm) | 20.21 | 20.15 |
| Pedicle diameter (mm) | 1.39 | 1.42 |
| Pedicle anthocyanin | n/a | RHS 181C |
| coloration | | Moderate red |
| Pedicle anthocyanin | Absent | Absent to very weak |
| intensity | | |
| Relative number of | 19.6 | 22.1 |
| pedicel prickles | Medium | Many |
| Peduncle anthocyanin | Absent | Present |
| presence | | |
| Peduncle anthocyanin | n/a | RHS 184A |
| coloration | | Greyish red |
| Peduncle anthocyanin | Absent | Absent to very weak |
| intensity | | |

TABLE 7
PEST AND DISEASE REACTIONS

| | | ‘MAJESTIC’ |
|---|---|---|
| Characteristic | ‘PS-12.082-54’ | (US PP31,716) |
| Spotted wing drosophila | Susceptible | Susceptible |
| (Drosophila suzukii) | | |
| Two spotted spider mite | Susceptible | Susceptible |
| (Tetranychus urticae) | | |
| Grey fruit mold | Susceptible | Susceptible |
| (Botrytis cinerae) | | |
| Powdery mildew | Moderately | Moderately |
| (Podosphaera aphanis var. aphonic) | susceptible | susceptible |
| Yellow rust | Moderately | Moderately |
| (Phragmidium rubi-idaei) | resistant | resistant |

We claim:

1. A new and distinct raspberry variety, as described and illustrated herein.
